(19) United States
(12) Patent Application Publication
(10) Pub. No.: US 2021/0134400 A1
(43) Pub. Date: May 6, 2021

(54) SELECTING A CRITERION FOR DETERMINING WHICH SUBJECTS TO INCLUDE IN A MEDICAL TRIAL

(71) Applicant: KONINKLIJKE PHILIPS N.V., EINDHOVEN (NL)

(72) Inventor: Monique Hendriks, Eindhoven (NL)

(21) Appl. No.: 16/486,938

(22) PCT Filed: Feb. 27, 2018
(86) PCT No.: PCT/EP2018/054726

§ 371 Date: Aug. 19, 2019

Related U.S. Application Data
(60) Provisional application No. 62/463,909, filed on Feb. 27, 2017.

(51) Int. Cl. G16H 10/20; G16H 10/60

(52) U.S. Cl. CPC G16H 10/20; G16H 10/60

(57) ABSTRACT

According to an aspect, there is provided a method of selecting a criterion for determining which subjects from a plurality of subjects to include in a medical trial. The method comprises, for a dataset comprising one or more entries, for each of the plurality of subjects: obtaining a plurality of test criteria; determining, for each test criterion, a measure of how evenly the entries in the dataset are distributed between satisfying the test criterion and not satisfying the test criterion; and selecting a criterion from the plurality of test criteria based on the determined measures. A computer program product is also disclosed.

### TECHNICAL FIELD

Various embodiments described herein relate to methods and apparatus for selecting a criterion for determining which subjects from a plurality of subjects to include in a medical trial.

### BACKGROUND

Medical trials are only statistically robust if they have an appropriate number of participants. The number of patients that can be enrolled in a trial depends on various factors including i) the number of patients that are eligible for the trial ii) the number of those patients that are contacted/contactable to apply for the trial (i.e. the number of patients, or their doctors, that are aware of the existence of the trial) and iii) the number of patients that accept a place on the trial.

As healthcare and data management is modernized, the first two of these factors can be influenced more easily as large sets of patient records can be searched for eligible patients, and the eligible patients and/or their clinicians can be electronically notified of the existence of the trial. Such datasets may be large, containing data of many tens or hundreds of thousands of patients.

When designing a medical trial, a clinician may specify a set of criteria that a person should meet in order to be eligible to take part in the trial. For example, the clinician may specify an age range for the participants and/or one or more diseases that the patients should have in order to be eligible for the trial.

To create a trial of the desired size, (i.e. not too big or too small), clinicians investigate how loosening or restricting certain criteria might change the number of patients who are eligible for the trial. There are tools available that help the clinician to visualize the data and to help them determine which thresholds should be used to select an appropriate number of patients. These help to give the clinician insights into which criteria are the best candidates for reconsidering.

With the advent of big data, creating such visualizations becomes computationally inefficient due to the fact that every time the user changes a criterion, the entire set of calculations needs to be redone. On a big dataset, it can take too long to perform the calculations in real time which prevents clinicians from being able to gain insights by ‘playing’ with tightening and loosening different criteria.

Therefore new methods are needed to help clinicians explore how different criteria affect the sample sizes of their trials, particularly ones that can be applied to big datasets.

### SUMMARY

As described above, traditional data processing methods for exploring which patients to include in a medical trial become inefficient when the database of patients become particularly large. Furthermore, the results become increasingly difficult for clinicians and researchers to interpret. There is therefore a need for improved methods for exploring medical trial participation in large datasets.

According to various embodiments, there is provided a method of selecting a criterion for determining which subjects from a plurality of subjects to include in a medical trial, the method including: for a dataset comprising one or more entries for each of the plurality of subjects: obtaining a plurality of test criteria; determining, for each test criterion, a measure of how evenly the entries in the dataset are distributed between satisfying the test criterion and not satisfying the test criterion; and selecting a criterion from the plurality of test criteria based on the determined measures.

Selecting a criterion to relax or loosen based on a measure of how evenly entries in the dataset are distributed between satisfying a criterion and not satisfying the criterion can increase the number of subjects to be included in a medical trial by an appropriate number, in a quick and easy manner. The number of calculations to be performed is reduced compared to existing methods, so an amount of processing power expended is reduced. Further, a user can more easily visualise an effect of relaxing a particular criterion, than in an existing method.

In some embodiments, the measure may comprise an entropy of the dataset associated with how many subjects satisfy the test criterion and how many subjects do not satisfy the test criterion. The measure may comprise an expected reduction in an entropy of the dataset if the test criterion is applied to the dataset. In some embodiments, the measure includes an information gain.

The step of selecting may, in some embodiments, comprise determining whether to use a first test criterion from the plurality of test criteria based on a comparison of the determined measure for the first test criterion and the determined measure of each of the other criteria in the plurality of test criteria. The step of selecting may comprise selecting a second criterion as the criterion if the comparison indicates that applying the second criterion would result in a reduction in entropy of the dataset that is lower than a reduction in entropy resulting from an application of any of the other criteria in the plurality of criteria.

The step of selecting may comprise selecting a third criterion as the criterion if the measure indicates that applying the third criterion would result in a reduction in entropy that is lower than a defined threshold reduction in entropy.

In some embodiments, the step of selecting may comprise arranging the determined measures in an order according to numerical magnitudes of the determined measures. The step of selecting may comprise presenting a list of the plurality of test criteria to a user, the list being ordered according to said order.

The step of determining may comprise determining, for each test criterion, a first value indicative of a number of subjects that satisfy the test criterion and a second value indicative of a number of subjects that do not satisfy the test criterion. The method may further comprise, for each criterion in the plurality of test criteria, presenting, with said list, at least one of each first value and each second value.

In some embodiments, the method may comprise determining a test criterion to adjust from the plurality of test criteria, based on the determined measures; defining a plurality of adjusted criteria for the determined test criterion; and calculating the measure for each of the adjusted criteria. The step of selecting a criterion may comprise selecting an adjusted criterion from the plurality of adjusted criteria, based on the calculated measures for the adjusted criteria.

The method may, in some embodiments, comprise obtaining an indication that a particular test criterion cannot be adjusted. The step of determining, for each test criterion, a measure of how evenly the entries in the dataset are distributed between satisfying the test criterion and not satisfying the test criterion may comprise determining a subset of data values that satisfy the particular test criterion; and determining, for each test criterion other than the particular test criterion, a measure of how evenly the entries in the subset of data values are distributed between satisfying the test criterion and not satisfying the test criterion.

The step of determining a test criterion from the plurality of test criteria to adjust may comprise selecting a criterion that has one of a highest measure; or a lowest measure.

One of the plurality of test criteria may comprise a defined range within which an entry is to fall for the subject associated with the entry to be included in the medical trial. In some embodiments, the test criteria may comprise a requirement which an entry is to satisfy for the subject associated with the entry to be included in the medical trial.

According to some embodiments, there is provided a computer program product comprising a non-transitory computer readable medium, the computer readable medium having computer readable code embodied therein, the computer readable code being configured such that, on execution by a suitable computer or processor, the computer or processor is caused to perform the method of any of the preceding claims.

### BRIEF DESCRIPTION OF THE DRAWINGS

For a better understanding, and to show more clearly how it may be carried into effect, reference will now be made, by way of example only, to the accompanying drawings, in which:

FIG. 1 is a table of an exemplary dataset containing entries for a plurality of subjects;

FIG. 2a is a decision tree showing how a set of criteria can be used to select subjects for a medical trial;

FIG. 2b is an expanded decision tree showing how the number of participants in a medical trial may be changed by changing an age criterion;

FIG. 3 is a schematic illustration of an example apparatus according to embodiments;

FIG. 4 is a flowchart of an example method according to embodiments; and

FIG. 5 is a flowchart of a further example method according to embodiments.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term “or” refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

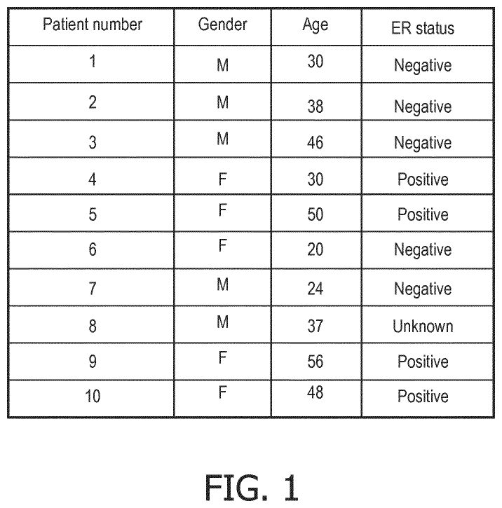

FIG. 1 is a table showing example patient records for ten patients. Each record contains the patient's gender, age and ER STATUS (estrogen receptor status). The ER status can have values of “positive”, “negative” or “unknown”. When designing a medical trial, a clinician will specify a set of test criteria, which are criteria that the clinician is considering for use in defining which patients are to be included in the medical trial. For example, the clinician may start by considering patients that are female, younger than 45 with ER status equal to positive. In this example, there are thus three test criteria:

Criterion1: Gender=Female

Criterion2: Age<45

Criterion3: ER status=positive.

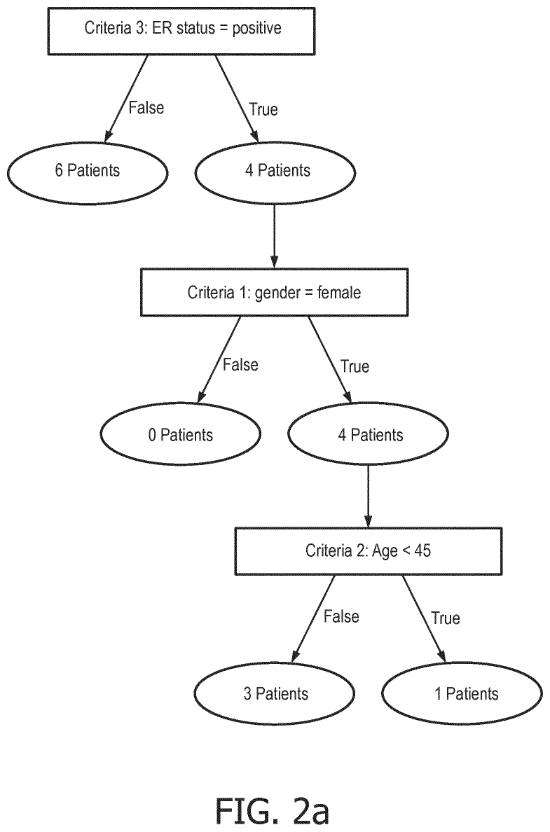

A patient must satisfy all three criteria to be included in the medical trial. In this example, only one patient from the 10 patients in Table 1 satisfies the test criteria. If the clinician wants more than one patient in the medical trial, then they will need to adjust (in this case loosen) the criteria so that more patients can be added to the sample. Existing software tools enable a clinician to visualise a dataset and determine which criteria to loosen based on certain visualisations. One such way of visualising the dataset in FIG. 1 is shown in FIG. 2a which shows a decision tree showing the numbers of patients that are included and excluded due to each criterion. For clarity, it is noted that the criteria in the decision tree can be in any order. The embodiments herein provide a way to construct the best order in which to consider loosening criteria. To help the clinician visualise the effects of loosening the criterion, the decision tree may be expanded as shown in FIG. 2b. FIG. 2b shows the number of patients in different age ranges to provide an illustration of how the number of patients can be changed by changing the age criterion. On the basis of the expanded decision tree, the clinician can see, for example, that extending the upper age limit to 50 results in one additional patient, and extending the upper age limit to 55 results in two additional patients. Generating decision trees in this way for every criterion and every possible order of criteria (from top to bottom) becomes increasingly computationally expensive as more patients are added to the dataset and/or more complex criteria are used. Furthermore, as the complexity increases, it becomes difficult (if not impossible) for clinicians to interpret all of the possible options for loosening all criteria.

In examples where there are more criteria and many more patients, the decision tree quickly becomes complex to the point where it is difficult for a clinician to interpret. Furthermore, each time the clinician changes one or more of the criteria, the numbers in each branch need to be recalculated. When big data is involved, for example involving upward of hundreds of thousands of database entries, the database queries required to compute the decision tree become prohibitively slow to execute in real time. There is thus a need to provide new tools to help clinicians explore appropriate criteria for use in selecting patients to be invited to participate in medical trials.

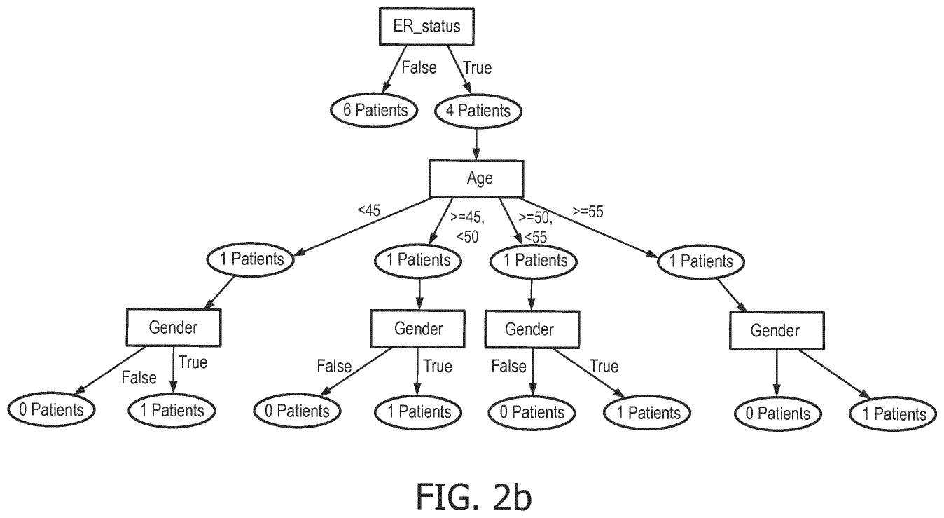

FIG. 3 shows an apparatus 2 according to embodiments of the present disclosure, for determining which subjects from a plurality of subjects to include in a medical trial. In the examples that follow, the term ‘subject’ is used interchangeably with ‘patient’, to indicate a person who may be considered for inclusion in the trial. The apparatus 2 includes a processing unit 4 that is in communication with a database 6 which holds a dataset including information about a plurality of subjects. The processing unit 4 can query the dataset held on a database 6 and process the resulting data to determine which subjects from a plurality of subjects to include in a medical trial.

In some embodiments, the apparatus 2 is a computing device, such as a laptop, a desktop computer, a smartphone, a tablet computer or some other portable electronic device. The database 6 may be contained within the apparatus 2 or may be remote from the apparatus 2, for example, the database 6 may be stored on a remote server. Queries run by processing unit 4 on the database 6 may therefore be executed locally in the apparatus 2, or remotely.

The processing unit 4 can be implemented in numerous ways, with software and/or hardware, to perform the various functions described below. The processing unit 4 may comprise one or more microprocessors or digital signal processor (DSPs) that may be programmed using software or computer program code to perform the required functions and/or to control components of the processing unit 4 to effect the required functions. The processing unit 4 may be implemented as a combination of dedicated hardware to perform some functions (e.g. amplifiers, pre-amplifiers, analog-to-digital convertors (ADCs) and/or digital-to-analog convertors (DACs)) and a processor (e.g., one or more programmed microprocessors, controllers, DSPs and associated circuitry) to perform other functions. Examples of components that may be employed in various embodiments of the present disclosure include, but are not limited to, conventional microprocessors, DSPs, application specific integrated circuits (ASICs), and field-programmable gate arrays (FPGAs).

In various implementations, the processing unit 4 may be associated with or comprise one or more memory units 8 such as volatile and non-volatile computer memory such as RAM, PROM, EPROM, and EEPROM. The processing unit 4 or associated memory unit 8 can also be used for storing program code that can be executed by a processor in the processing unit 4 to perform the method described herein. The memory unit 8 can also be used to store data retrieved from the database 6.

It will be understood that FIG. 3 constitutes, in some respects, an abstraction and that the actual organization of the components of the apparatus 2 may be more complex than illustrated. Furthermore, the apparatus 2 may comprise additional components not specifically illustrated in FIG. 3, for example, apparatus 2 may comprise one or more devices for enabling communication with a user such as a researcher or clinician. For example, the apparatus 2 may include a display, a mouse, and/or a keyboard for receiving user commands. It is noted that the terms user, clinician and researcher may be used interchangeably in the examples herein.

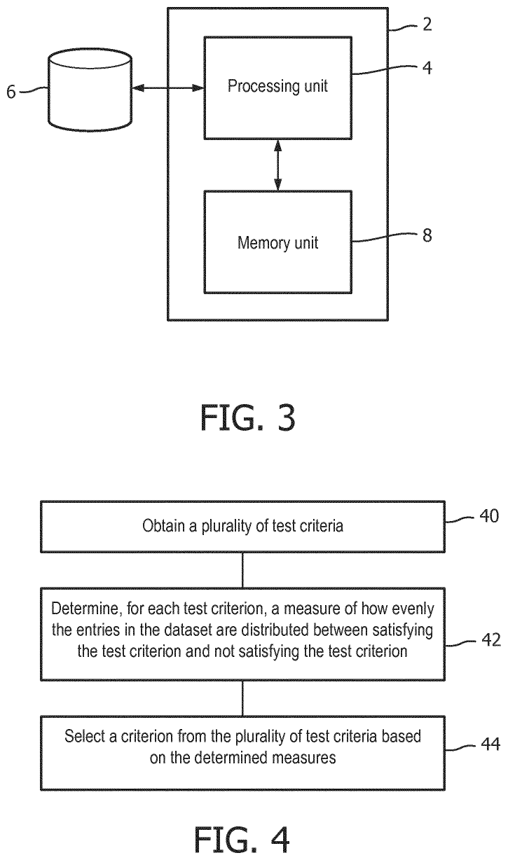

FIG. 4 shows a flowchart representing a method of selecting a criterion for determining which subjects from a plurality of subjects to include in a medical trial. The method can be performed by the apparatus 2, and in particular by the processing unit 4. The method is performed on a dataset including one or more entries for each of the plurality of subjects. As described above, the dataset can be stored locally on apparatus 2, or be stored remotely, for example on a remote server. The dataset may comprise a record for each subject containing one or more fields, each field containing information about the subject. Examples of fields include, but are not limited to, the age, gender and location of the subject, and whether the subject has a disease, such as, for example, heart disease, diabetes, high cholesterol, or cancer. Some fields may contain more detailed information such as for example, tumour size, or the stage of advancement of a tumour.

In a first step 40, the method includes obtaining a plurality of test criteria. This step can comprise the processing unit 4 receiving the plurality of test criteria as input by a user, for example from a clinician, or obtaining (e.g. retrieving) the test criteria from a memory unit 8 or receiving the plurality of test criteria from a remote computer or server.

Each test criterion represents a test that can be used to decide whether a subject should be included or excluded from the trial. Criteria can be based on any characteristic of the subject, such as the gender, age, and location of the subject, or whether the subject has a disease or condition, such as high blood pressure, heart disease, diabetes, cancer or the like. A criterion can be of two forms:
Categorical: e.g. “the patient must be female”; “the patient must have a HER2 positive tumour”; or “the patient must be Caucasian”.
Numerical (either on a continuous or discrete scale): e.g. “the patient must be older than 18 and younger than 50”; “the tumour size must be less than 1 cm in diameter”.

For criteria based on fields in the dataset containing categorical data, a criterion needs to be generated relating to a field in the dataset, based on the levels that the field may take (e.g. male or female, HER2 positive, HER2 negative, or unknown HER2 status, a list of possible races and so on). When considering numerical fields, a criterion needs to be generated where the levels are a certain range of the variable, e.g. 30<age <45. Each criterion may have two possible outcomes: a patient either satisfies the criterion or does not satisfy the criterion. For example, if only males are included, the criterion may have the possible outcomes ‘male’ and ‘not male’; if only patients younger than 50 are to be included, the criterion may have the possible outcomes ‘younger than 50’ and ‘50 and older’.

Other examples of possible criteria are given in the examples above and below.

In a second step 42, the method includes determining, for each test criterion, a measure of how evenly the entries in a dataset are distributed between satisfying the test criterion and not satisfying the test criterion. In some embodiments, the measure is a measure of the entropy associated with how many subjects satisfy the test criterion and how many subjects do not satisfy the test criterion. In some embodiments the measure is a measure of the expected reduction in an entropy of the dataset if the test criterion is applied to the dataset. In some embodiments, the measure may be the information gain associated with applying the criterion.

The information gain of a criterion is defined in terms of entropy. Suppose we have a dataset S and observed classifications 1 . . . c, then entropy is a measure of how well the data is balanced over the different classifications. For example, if there are two classes, a perfect balance (each class has an equal number of observations), results in entropy=1; if only one of the two classes is present in the data (extremely unbalanced), then entropy=0. So a balanced dataset has a high entropy and an unbalanced dataset has a low entropy. In the examples herein, there are two classes because each subject is classed as either satisfying the criterion (class 1) or not satisfying the criterion (class 2). In situations where there are two classes, the entropy varies between 0 and 1. In other applications where there are more classes, the entropy may be >1.

Entropy is calculated as follows:

$$Entropy ( S ) = {\sum }_{i = 1}^{c} - {p}_{i} {log}_{2} {p}_{i}$$

where pᵢ is the proportion of observed i's in the dataset S.

The information gain of a criterion A in the dataset S quantifies the expected reduction in entropy if we were to split the dataset according to criterion A.

The information gain of a criterion A from the dataset S is then defined as:

$$gain ( S , A ) = entropy ( S ) - {\sum }_{v ∈ values ( A )} \frac{\vert {S}_{v} \vert}{\vert S \vert} entropy ( {S}_{v} )$$

Where entropy(S) is the entropy of the entire dataset and

$${Σ}_{v ∈ values ( A )} \frac{\vert {S}_{v} \vert}{\vert S \vert} entropy ( {S}_{v} )$$

is the sum of the entropies of the subsets created by splitting by criterion v multiplied by the fraction of observations that belong to each subset. Values(A) is the set of all possible values for criterion A, Sᵥ is the subset of observations from S that have value v for criterion A.

In a third step 44, the method includes selecting a criterion from the plurality of test criteria based on the determined measures. In some embodiments, selecting the criteria includes ranking the test criteria in ascending or descending order according to the magnitudes of the measures of the criteria and selecting a criterion based on the ranking.

For example, in a scenario where the measure is the information gain of a criterion, a higher number of subjects can be gained by loosening a criterion that has a higher information gain than can be gained by loosening a criterion that has a lower information gain. Thus, if a larger sample is needed, then a criterion may be selected that has a high information gain, whereas if only a small number of additional participants are required, then conversely a criterion with a low information gain may be selected.

Thus, in some embodiments, the method of selecting a criterion includes determining whether to use a first test criterion from the plurality of test criteria based on a comparison of the determined measure for the first test criterion and the determined measure of each of the other criteria in the plurality of test criteria.

In some embodiments, a criterion may be chosen if it has the lowest information gain. This indicates that applying the selected criterion would result in a reduction in entropy of the dataset that is lower than a reduction in entropy resulting from an application of any of the other criteria in the plurality of criteria.

Alternatively still, the measure may be compared to a threshold. For example, a criterion may be chosen if applying that criterion would result in a reduction in entropy that is lower than a defined threshold reduction in entropy.

In some embodiments, the criteria may be presented to a user, such as a clinician in order of their information gain, to provide the clinician with an indication of which criteria may be the best to consider.

Generally, when investigating trial feasibility, criteria having a higher information gain yield more interesting and useful opportunities for loosening (i.e. loosening a criterion with a relatively higher information gain would result in a relatively larger increase in the number of subjects to be included in the medical trial than a relatively lower information gain). Criteria with low information gains might be less interesting, as these might increase the number of eligible subjects/patients by only small increments. In some cases, a criterion having a low information gain might be so restrictive (e.g. adding only one extra subject to the medical trial) that it is not useful at all to reconsider and thus can quickly be discarded.

The advantage of this method over the visualization method described above, is that the calculations of information gain only have to be done once in order to inform the user of which criteria are optimal to increase sample sizes. Thus, instead of the clinician ‘blindly’ trying different criteria resulting in a large number of recalculations, or having to interpret a complex decision tree, an ordered list of criteria can be presented to the user.

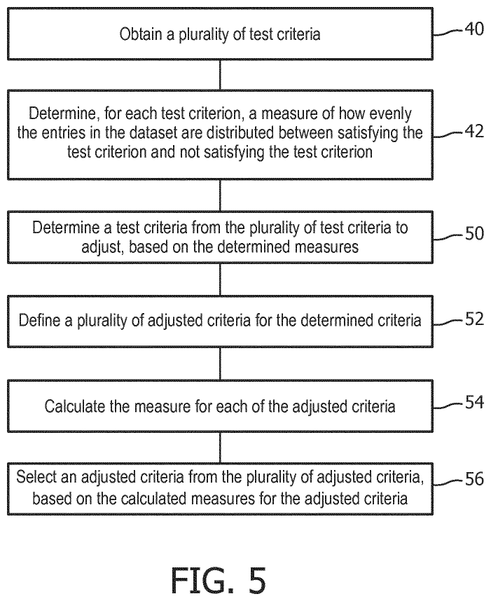

FIG. 5 shows another method according to an embodiment. In this embodiment, after the steps of obtaining a plurality of test criteria (step 40) and determining, for each test criterion, a measure of how evenly the entries in the dataset are distributed between satisfying the test criterion and not satisfying the test criterion (step 42), the method includes in step 50, determining a test criterion to adjust from the plurality of test criteria, based on the determined measures.

In some embodiments, the step of determining a test criterion to adjust includes comparing the measures of each criteria. If only a small number of additional participants are required, then step 50 includes determining to adjust a criterion for which the corresponding measure indicates that a small number of additional participants would be gained by changing that criterion. For example, if the measure is the information gain, then to increase the selected number of participants by a small amount, it is better to adjust a criterion with a low information gain than one with a high information gain. Conversely, if a large number of additional participants is required, then it is better to loosen a criterion with a high information gain as opposed to a low information gain. Considering the example discussed above with the data given in FIG. 1 the test criteria are:

Criterion1: Gender=Female

Criterion2: Age<45

Criterion3: ER status=positive

Using the information gain as the measure, the information gain for each criteria is (calculated using the formula above):

Information gain for criterion 1: 0.108031546146

Information gain for criterion 2: 0.0789821406003

Information gain for criterion 3: 0.144484343806

From these values, to provide the largest increase in participants, ER status would be the best candidate to consider to loosen because it has the largest value of the information gain.

Once it is determined which test criteria should be adjusted, the method includes, in a step 52, defining a plurality of adjusted (i.e. loosened) criteria for the determined criteria. The plurality of adjusted criteria represent possible alternative criteria that could be used to increase the number of participants. For example, the ER status can take values of positive, negative or unknown and therefore, the different possible ways of loosening the ER status are:

Adjusted criterion 1: ER status=positive or unknown

Adjusted criterion 2: ER status=positive or negative

Adjusted criterion 3: ER status=positive, negative or unknown.

For numerical criterion, such as age, it is not necessary to calculate every combination of possible ranges. For example, starting from a criterion of 35<age<45, it isn't necessary to compute every possible permutation of age ranges, such as 0<age<5; 5<age<15; 15<age<25 and so on, as it is more likely that the clinician will be interested in age ranges similar to the range in the starting criteria of 35<age<45. It is thus possible to assume that the loosening of a numerical criterion will always happen in ranges close to the initial range restriction. For example, if the inclusion criterion is that the patient needs to be in the age range 30 to 50, then it is more likely that the criterion will be loosened to ages 25 to 50 or 30 to 55, than is it to additionally include patients between 20 and 25 or patients between 55 and 60. In some embodiments, weights may be assigned to each range in decreasing order the further the range is away from the current inclusion criterion. This biases the results towards changes in range that are more likely to be of interest to the clinician.

Once the adjusted criteria are defined, step 54 includes calculating the measure for each of the adjusted criteria. This is done in the same way as described above (e.g. in step 42). The step of selecting a criterion (step 44) then includes selecting an adjusted criterion from the plurality of adjusted criteria, based on the calculated measures for the adjusted criteria (step 56). As described above, an adjusted criterion may be selected depending on how many additional participants are required. In the example where the measure is an information gain, if larger numbers of additional subjects are required, step 44 may comprise selecting an adjusted criterion that has a larger (or the largest) information gain, compared to a situation where only a few additional subjects are required, in which case step 44 may comprise selecting an adjusted criterion that has a small (or the smallest) information gain.

Thus, in this way, starting from an initial set (i.e. a plurality) of test criteria, the method provides a way of suggesting the criteria to consider investigating in order to incrementally change the sample size and then suggests appropriate adjustments to said criteria in order to achieve a change in sample size desired by the clinician. Thus instead of the clinician ‘blindly’ trying different criteria, the effort for the clinician is reduced by providing an ordered list of criteria, indicating which criteria are mathematically the best options to consider adjusting in order to obtain a desired sample size. Furthermore, the number of calculations that are performed is reduced, resulting in more efficient use of computational power.

Additionally, given that in the calculations the size of the different subsets S is used to calculate the information gain, the values for the sizes of each subset can be stored, so that the exact number of patients who can be added if a constraint is loosened can be presented to the user, thereby making recalculations after loosening the constraint unnecessary.

In a further embodiment, the method may comprise obtaining an indication that a particular test criterion cannot be adjusted. For example, it isn't desirable to include females in a study of prostate cancer, or to include under 30's in a study relating to ageing. Such an indication may be provided by a user, such as a clinician or researcher, and may be input by such a user in real time.

In this embodiment, the step of determining, for each test criterion, a measure of how evenly the entries in the dataset are distributed between satisfying the test criterion and not satisfying the test criterion (step 42) includes determining a subset of data values that satisfy the particular test criterion (i.e. the criterion that has been indicated as not being capable of being adjusted). The measure of how evenly the entries in the subset of data values are distributed between satisfying the test criterion and not satisfying the test criterion is then calculated only for the subset that satisfies the criterion that cannot be loosened.

As described in the examples above, in some embodiments, the step of determining a test criterion from the plurality of test criteria to adjust includes selecting a criterion that has a high measure compared to the other test criteria, or the highest measure if lots of additional subjects are required, or a low, or lowest measure if just a few are required.

This can be illustrated in the context of the example described above with respect to FIG. 1. Based on the information gain of the three criteria, it was determined that ER status was the best criteria to consider loosening. Suppose, however, that the clinician indicates that the restriction on ER status definitely cannot be loosened for the purposes of their trial. Based on the three information gain values, one might be inclined to choose Gender as the next candidate criterion for loosening. However, when the information gains for Age and Gender are recalculated given that the ER status criterion cannot be relaxed, one arrives at the following:

Information gains of subset with ER status=positive:
Gender: 0.0
Age: 0.811278124459

Therefore, the clinician would be better to consider adjusting the age range of participants. This makes sense from the data in table 1: if Gender had been chosen to be relaxed, it would result in no more patients being added to the sample, even if men were included.

Variations to the disclosed embodiments can be understood and effected by those skilled in the art in practicing the principles and systems disclosed herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A computer implemented method of selecting a criterion for determining which subjects from a plurality of subjects to include in a medical trial, the method comprising:
for a dataset comprising one or more entries for each of the plurality of subjects;
obtaining a plurality of test criteria, a test criterion being based on a characteristic of the subject, a test criterion being a categorical or numerical criterion;
determining, for each test criterion, a measure of how evenly the entries in the dataset are distributed between satisfying the test criterion and not satisfying the test criterion; and
selecting a criterion from the plurality of test criteria based on the determined measures, the step of selecting comprising arranging the determined measures in an order according to numerical magnitudes of the determined measures, and presenting a list of the plurality of test criteria to a user, the list being ordered according to said order.

2. A method as in claim 1 wherein the measure comprises an entropy of the dataset associated with how many subjects satisfy the test criterion and how many subjects do not satisfy the test criterion.

3. A method as in claim 1 wherein the measure comprises an expected reduction in an entropy of the dataset if the test criterion is applied to the dataset.

4. A method as in claim 1 wherein the measure comprises an information gain.

5. A method as in claim 1 wherein the step of selecting comprises determining whether to use a first test criterion from the plurality of test criteria based on a comparison of the determined measure for the first test criterion and the determined measure of each of the other criteria in the plurality of test criteria.

6. A method as in claim 5 wherein the step of selecting comprises selecting a second criterion as the criterion if the comparison indicates that applying the second criterion would result in a reduction in entropy of the dataset that is lower than a reduction in entropy resulting from an application of any of the other criteria in the plurality of criteria.

7. A method as in claim 1 wherein the step of selecting comprises selecting a third criterion as the criterion if the measure indicates that applying the third criterion would result in a reduction in entropy that is lower than a defined threshold reduction in entropy.

8. (canceled)

9. A method as in claim 1, wherein the step of determining comprises determining, for each test criterion, a first value indicative of a number of subjects that satisfy the test criterion and a second value indicative of a number of subjects that do not satisfy the test criterion; and wherein the method further comprises:
for each criterion in the plurality of test criteria, presenting, with said list, at least one of each first value and each second value.

10. A method as in claim 1 further comprising:
determining a test criterion to adjust from the plurality of test criteria, based on the determined measures;
defining a plurality of adjusted criteria for the determined test criterion; and
calculating the measure for each of the adjusted criteria;
wherein the step of selecting a criterion comprises selecting an adjusted criterion from the plurality of adjusted criteria, based on the calculated measures for the adjusted criteria.

11. A method as in claim 10 further comprising:
obtaining an indication that a particular test criterion cannot be adjusted;
wherein the step of determining, for each test criterion, a measure of how evenly the entries in the dataset are distributed between satisfying the test criterion and not satisfying the test criterion comprises:
determining a subset of data values that satisfy the particular test criterion; and
determining, for each test criterion other than the particular test criterion, a measure of how evenly the entries in the subset of data values are distributed between satisfying the test criterion and not satisfying the test criterion.

12. A method as in claim 10 wherein the step of determining a test criterion from the plurality of test criteria to adjust comprises selecting a criterion that has one of:
a highest measure; or
a lowest measure.

13. A method as in claim 1 wherein one of the plurality of test criteria comprises a defined range within which an entry is to fall for the subject associated with the entry to be included in the medical trial.

14. A method as in claim 1 wherein the test criteria comprises a requirement which an entry is to satisfy for the subject associated with the entry to be included in the medical trial.

15. A computer program product comprising a non-transitory computer readable medium, the computer readable medium having computer readable code embodied therein, the computer readable code being configured such that, on execution by a suitable computer or processor, the computer or processor is caused to perform a computer-implemented method of selecting a criterion for determining which subjects from a plurality of subjects to include in a medical trial, the method comprising:
for a dataset comprising one or more entries for each of the plurality of subjects;
obtaining a plurality of test criteria, a test criterion being based on a characteristic of the subject, a test criterion being a categorical or numerical criterion;
determining, for each test criterion, a measure of how evenly the entries in the dataset are distributed between satisfying the test criterion and not satisfying the test criterion; and selecting a criterion from the plurality of test criteria based on the determined measures, the step of selecting comprising arranging the determined measures in an order according to numerical magnitudes of the determined measures, and presenting a list of the plurality of test criteria to a user, the list being ordered according to said order.

16. A computing device for determining which subjects from a plurality of subjects to include in a medical trial, the computing device comprising:
a processing unit in communication with a database, the database holding a dataset including information about a plurality of subjects, the processing unit being configured to:
obtain a plurality of test criteria, a test criterion being based on a characteristic of the subject, a test criterion being a categorical or numerical criterion;
determine, for each test criterion, a measure of how evenly the entries in the dataset are distributed between satisfying the test criterion and not satisfying the test criterion; and
select a criterion from the plurality of test criteria based on the determined measures, selecting the criterion comprising arranging the determined measures in an order according to numerical magnitudes of the determined measures, and presenting a list of the plurality of test criteria to a user, the list being ordered according to said order.
